(19) United States
(12) Patent Application Publication
(10) Pub. No.: US 2013/0210075 A1
(43) Pub. Date: Aug. 15, 2013

(54) ENHANCED PROTEIN EXPRESSION

(71) Applicants: Makkapati Satakarni, Secunderabad (IN); S. Nikam Vaibhav, Kolhapur (IN); Subrahmanyam Satyam, Hyderabad (IN)

(72) Inventors: Makkapati Satakarni, Secunderabad (IN); S. Nikam Vaibhav, Kolhapur (IN); Subrahmanyam Satyam, Hyderabad (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/520,850

(22) PCT Filed: Jan. 6, 2011
(86) PCT No.: PCT/US11/20367

§ 371 Date: Dec. 18, 2012

(30) Foreign Application Priority Data
Jan. 7, 2010 (IN) 48/CHE/2010

(51) Int. Cl. C07K 16/00

(52) U.S. Cl. CPC C07K 16/00
USPC 435/69.6; 435/69.1

(57) ABSTRACT

The application describes methods to enhance protein production using mammalian cells. Increased production of heterologous protein can be obtained by increasing osmolality and lowering temperature. The method maintains the cell growth rate and provides high product yield.

### INTRODUCTION

Aspects of the application relate to methods for enhancing heterologous protein production by mammalian cells. In specific embodiments, the application includes cell culture processes, wherein cells are fed in a profile mode to obtain optimum growth, followed by osmotic stress and lowering of temperature to obtain high product yield.

The use of recombinant DNA technology products for treatment of medical conditions has changed the face of the therapeutic industry. Therapeutic proteins are used to relieve patients suffering from various ailments such as cancers (monoclonal antibodies and interferons), heart attacks, strokes, cystic fibrosis, Gaucher's disease (enzymes and blood factors), diabetes (insulin), anaemia (erythropoietin), and haemophilia (blood clotting factors). Significant therapeutic benefits have been attained using these biopharmaceuticals, leading to high demand for these products. Hence, large scale, cost-effective and efficient manufacturing processes are required for their production. There remain significant challenges in the development of cost effective and efficient industrial scale production of recombinant proteins. Thus the manufacturing of biomolecules is a challenge, in particular, maintaining high viable cell density and prolonged cell culture lifetime of cell lines that express biomolecules.

Studies suggest that economical production of recombinant proteins in a cell culture production process can be brought about by either increasing the specific production capability of the cells or by increasing the time integral of viable cell concentration. To attain this, optimization of parameters that influence protein production, cell viability, and product yield is helpful. These include several physico-chemical factors such as pH buffering, partial pressure of oxygen (pO₂), partial pressure of carbon dioxide (pCO₂), temperature, and osmolality. Depending upon cell types, these parameters are adjusted for optimal growth; for instance, mammalian cells especially require pH 7.0-7.4 and a temperature range of 35-37° C. (J. Mather and D. Barnes, Animal Cell Culture Methods: Methods in Cell Biology, Academic Press, San Diego, 1998; Vol 57).

The optimization of these parameters to improve productivity in cell culture includes methods such as altering osmolality (by addition of salts and nutrients) during production, decreasing temperatures during specific phases of a cell culture, and/or changes in pH and pCO₂. Though these methods have been utilized to improve product yield, variability in the results have been noticed. For instance, studies by Kim et al. show that use of hyperosmolar medium does not improve the maximum antibody concentration substantially (Kim, M. S.; Kim, N. S.; Sung, H. S.; Lee, G. M.; In Vitro Cellular & Developmental Biology—Animal, 2002; 38(6): 314-319), while U.S. Pat. No. 6,238,891 reports otherwise. Thus, there remains a need in the art to continually improve yields.

Of all the process parameters mentioned, high osmolality (high osmotic pressure) is believed to be a factor that adversely affects cell growth (Kimura, R.; Miller, W. M. M, Biotechnol. Bioeng. 1996; 52: 152-160 and Kim, N. S.; Lee, G. M. J., Biotechnol. 2002; 95(3): 237-248). Ozturk et. al report that high osmolar conditions do not bring about a substantial increase in the final antibody titer, as the increase in antibody production rate may not be adequate to compensate for the slower growth rate (Ozturk, S. S.; Palsson, B. O.; Biotechnol. and Bioeng. 1991; 37: 989-993). Furthermore, studies in continuous cultures suggest that there is no stimulation of antibody production at higher levels (i.e., 425 mOsm/kg and above) (Cherlet, M.; Marc, A., Cytotechnol. 1999; 29: 71-84). However, U.S. Pat. No. 6,238,891 describes a method of improving monoclonal antibody (MAb) yields by culturing Chinese hamster ovary (CHO) cells in an environment of solute stress. Moreover, although the results with respect to heterologous protein production appear to vary with rCHO cells and, though increased osmolality, results in increased specific protein productivity, the final product yield may or may not improve (Ryu, J. S., Biotechnol. Bioeng. 2000; 70(2): 167-175). Thus, the effect of osmolality on the final antibody titer cannot be generalized.

Similarly, the effect of temperature as a parameter for cell growth and product yield has been diverse. This is attributed to variations in specific protein productivity, cell viability, and cell growth rate. Satoshi et. al. have reported improvements in cell viability and increase in monoclonal antibody production in CHO culture using temperature downshift (Satoshi, O.; Hiroyuki, S.; Masayoshi, T.; Haruhiko, T., Cyotechnol, 2006; 52(3): 192-207). Also, Yoon et. al. reported that beneficial effects of lowering temperature below 37° C. (for instance 33° C.) are mainly due to enhanced specific heterologous protein production and not solely due to improved culture longevity (Yoon, S. K.; Song, J. Y.; Lee, G. M.; Biotechnol. Bioeng. 2002; 82(3): 289-298). A difference here is noted with respect to specific protein production. Though lowering the temperature decreases the specific growth rate of cells, the specific protein production is variable. The specific productivity may or may not increase with lowering of temperatures. Thus the effect of lowering of temperature also varies with cell types and target protein (Yoon, S. K.; Song, J. Y.; Lee, G. M.; Biotechnol. Bioeng. 2002; 82(3): 289-298).

Apart from the process parameters mentioned above, feeding strategy can also play an important role in increasing product yield. The transition from batch cultures to fed batch, based on the need for superior productivity has been perceptible. During batch phase culture of recombinant cells, nutrients become limiting, leading to a reduction in cell performance (measured by cell viability, viable cell density, and protein yield). To overcome these effects, batch cultures are fed with a concentrated solution of medium and/or amino acids, a process known as fed batch culture. The strategy is used to achieve high cell densities but the addition of a highly concentrated feed solution to avoid dilution is challenging as it changes the culture osmolality and which in turn may lead to depressed cell growth.

Since final product yield is determined by cell culture longevity and specific protein secretion rate, fed-batch process development strategies aim at maximizing these parameters in order to attain higher yield. Though this has been studied extensively using osmolality or temperature shifts, but a concrete process is still awaited. Optimization of cell culture process by increased osmolality or reduced temperature remains a significant challenge, with the outcome far from certain. Further, combinations of process changes may result in extremely depressed cell growth which in turn may affect protein yield.

### SUMMARY

Aspects of the application provide methods to enhance heterologous protein production in mammalian cells is described here. Embodiments of methods involve growing cells in a medium and subjecting them to: i) increased osmolar values; and ii) lowering of temperature.

### BRIEF DESCRIPTION OF THE DRAWINGS

FIG. 1 is an illustration of effects of temperature and osmolality shifts on final antibody yields, as described in Examples 1-6.

FIG. 2 is an illustration effects of temperature and osmolality shifts on the cell viability during culture processes, as described in Examples 1-6.

FIG. 3 is an illustration of normalized antibody yields over the age of culture, as described in Examples 1-6.

### DETAILED DESCRIPTION

The term “osmolality” as used herein is defined as a measure of the osmoles of solute per kilogram of solvent (osmol/kg).

Integral of viable cells used herein refers to cell growth over time or integral of viable cells with respect to culture time that is used for calibration of specific protein production. The integral of viable cell concentration can be increased either by increasing the viable cell concentration or by lengthening the process time.

One of the goals of recombinant protein production is the optimization of culture conditions so as to obtain high levels of productivity. With a high demand for these products, even small incremental increases in productivity can be economically significant.

Optimization of cell process parameters to attain high productivity has been attempted in the past. Of all the parameters, increased osmolality and lowering of temperature have been studied extensively, but variability in results is evident.

Osmolality is an important physico-chemical factor in mammalian cell culture that affects cell growth and protein production. Cell cultures for recombinant protein production are typically maintained in a range of 320-380 mOsm/kg. Any marked change in osmolality depresses cell growth and reduces cell densities. This, in turn, leads to lessened time integral of viable cells, thereby lesser yield. The depressed cell growth may be a result of induced apoptotic cell death which might affect protein recovery. However, U.S. Pat. No. 6,238,891 suggests the use of increased osmolality (through addition of salts) to improve product yield.

Temperature is another important physico-chemical parameter that affects cell growth and production of proteins. Mammalian cell cultures are grown at a temperature of 37° C., simulating the human body temperature. A decrease in temperature leads to reduced growth rates, thereby increasing the generation time, which in turn affects protein production. However, beneficial effects have been reported by some studies. Lowering of temperature extends cell culture viability as it delays onset of apoptosis (Alison, M. et al.; Cytotechnol., 1997; 23: 47-54). Decreases in temperature may lead to: i) higher and longer cell viability (Furukawa, K.; Ohsuye, K.; Cytotechnol., 1998; 26: 13-164); ii) reduction in specific oxygen uptake; iii) decreased protease activity (Chuppa, S.; Tsai, Y. S.; Yoon, S.; Shackelford, S.; Rozales, C.; Bhat, R,; Tsay, G.; Matangiuhan, C.; Konstantinov, K.; Naveh, D.; Biotechnol. Bioeng. 1997; 55: 328-338); iv) improved tolerance against shear stress (Ludwig, A.; Tomeczkowski, J.; Kretzmer, G.; Apl. Microbio. Biotechnol. 1992; 38: 323-327); v) suppressed medium consumption; and/or vi) suppressed release of impurities from the cells.

Though lowering of temperature may have beneficial effects, these effects vary among cultures, with an increase in specific protein production being observed for some while others are unaffected. The final product yield is the net result of effect of temperature down shift on specific protein productivity, cell viability, cell growth, and cell death rate. Hence, the final product yield varies depending upon cell types and proteins of interest.

Feeding strategy may also affect the protein production. Osmolar effects by feeding of nutrients can bring about depressed cell growth. One method described in this application to avoid osmolar effects and to compensate for nutrient depletion is by addition of nutrients in a “profile mode.” The cells are subjected to a gradual increase in osmolality by addition of nutrients. The strategy involves a process, wherein the culture initially passes through a cell proliferation phase to generate a sufficiently high viable cell mass, while being subjected to gradual increase in osmolality (to reduce the effects of high osmolar conditions in a production phase). The cells are then subjected to increased osmolar conditions which enhance protein production and cells are maintained viable and productive without significant cell proliferation, leading to an increased time integral of viable cells and product yield. Maintenance of high cell density and viability during the production provides for high cell protein yield. Feeding in a “profile mode” brings about a gradual increase in osmolality by feeding of nutrients. However, the nutrient feeding strategy is designed in a manner that the osmolality increase does not slow down cellular growth.

An alternative way of increasing protein production is by addition of nutrients and/or salts after attaining a required cell density. The production phase involves a rapid, as opposed to a more gradual, increase in osmolality, thereby inducing stress and product expression.

With rising demands of the market, the lack of a clear process for higher product expression is evident. The present application provides methods to overcome the problem. The application provides strategies to enhance heterologous protein production from cells, using high osmolality and lowering of temperature. In particular embodiments, the application describes a process to enhance the monoclonal antibody expression in recombinant mammalian cells, more specifically Chinese hamster ovary (CHO) cells, by culturing cells to achieve optimal growth and then subjecting mammalian cells to a lower temperature along with high osmotic conditions.

In aspects, the present application provides methods for expression of protein in cells cultured in a medium, comprising:

(a) growing cells at one osmolar value and temperature; and

(b) subjecting cells to at least one higher osmolar value and a reduction in temperature, followed by harvesting.

In specific embodiments, the application provides high level production of proteins by first culturing cells at temperatures about 35-37° C. and an osmolality range of about 320-380 mOsm/kg, followed by subjecting the cells to increased osmolality of about 420 mOsm/kg and lowering of temperatures by about 2-7° C.

In specific embodiments, the application provides high level production of proteins by first culturing cells at temperatures about 35-37° C. and an osmolality range of about 320-380 mOsm/kg, followed by subjecting the cells to increased osmolality of ≧450 mOsm/kg and lowering of temperatures by about 2-7° C.

In an aspect, the application provides methods for expression of protein in cells cultured in a medium, embodiments comprising:

(a) growing cells at one osmolar value and temperature;

(b) culturing cells in at least one higher osmolar value; and

(c) subjecting cells to at least one increased osmolar value and reduction in temperature, followed by harvesting.

In embodiments, the application provides methods for high level production of mammalian, human, or murine monoclonal antibodies by first culturing cells at temperatures about 35-37° C. and osmolality about 320 mOsm/kg, followed by culturing the cells at a higher osmolality range of about 380-420 mOsm/kg by gradual addition of nutrients, followed by subjecting cells to further increased osmolality ≧450 mOsm/kg and lowering of temperatures by about 2-7° C.

In embodiments, the application provides methods for expression of protein by growing cells at about 37° C. and osmolality about 320 mOsm/kg, followed by culturing cells at an increased osmolar value about 410 mOsm/kg, and further followed by subjecting cells to an increased osmolar value ≧450 mOsm/kg and lowering of temperature to about 33° C.

In an aspect, the application provides methods for high protein expression in a cell culture medium, embodiments comprising:

a) growing cells at an osmolar value; followed by

b) culturing cells in at least one higher osmolar value;

c) subjecting cells to at least one increased osmolar value and reduction in temperature;

d) subjecting cells to further increased osmolar value; and

e) recovering the protein from the culture.

In embodiments, the application provides methods for expression of protein by growing cells at about 37° C. and osmolality about 310-330 mOsm/kg, followed by culturing cells at an increased osmolar value about 400-420 mOsm/kg, and further followed by subjecting cells to an increased osmolar value about 450-470 mOsm/kg and lowering of temperature to about 33° C., followed by subjecting cells to an increased osmolar value ≧480 mOsm/kg.

In embodiments, the application provides methods for expression of protein by growing cells at about 37° C. and osmolality about 320 mOsm/kg, followed by culturing cells at an increased osmolar value about 410 mOsm/kg, and further followed by subjecting cells to an increased osmolar value about 460 mOsm/kg and lowering of temperature to about 33° C., followed by subjecting cells to an increased osmolar value about 480 mOsm/kg.

Cell culture media that are useful in the application include, but are not limited to, the commercially available products PF CHO (HyClone®), PowerCHO® 2 (Lonza), Zap-CHO (Invitria), CD CHO, CDOptiCHO™ and CHO-S-SFMII (Invitrogen), ProCHO™ (Lonza), CDM4CHO™ (Hyclone), DMEM (Invitrogen), DMEM/F12 (Invitrogen), Ham's F10 (Sigma), Minimal Essential Media (Sigma), and RPM)-1640 (Sigma).

Certain specific aspects and embodiments of the application are more fully described by the following examples. These examples should not, however, be construed as limiting the scope of the application.

### Example 1

An anti-VEGF antibody was cloned and expressed in a CHO cell line as described in U.S. Pat. No. 7,060,269, which is incorporated herein by reference. rCHO cells expressing antibody at a seeding density of 0.2-0.6 million cells/mL were grown in a PF CHO (HyClone®, Catalog no. SH30335 and SH30334) at 37° C. and an initial osmolality of 380-390 mOsm/kg. On attainment of optimum cell growth (IVCC of 3 to 12 million cell-days/mL), the osmolality was increased to about 420 mOsm/kg and the temperature was lowered to 33° C. The culture was finally harvested after 288 hours or at 550% viablility. The resulting antibody yield was determined.

The antibody yield and cell viability are disclosed in Table 1.

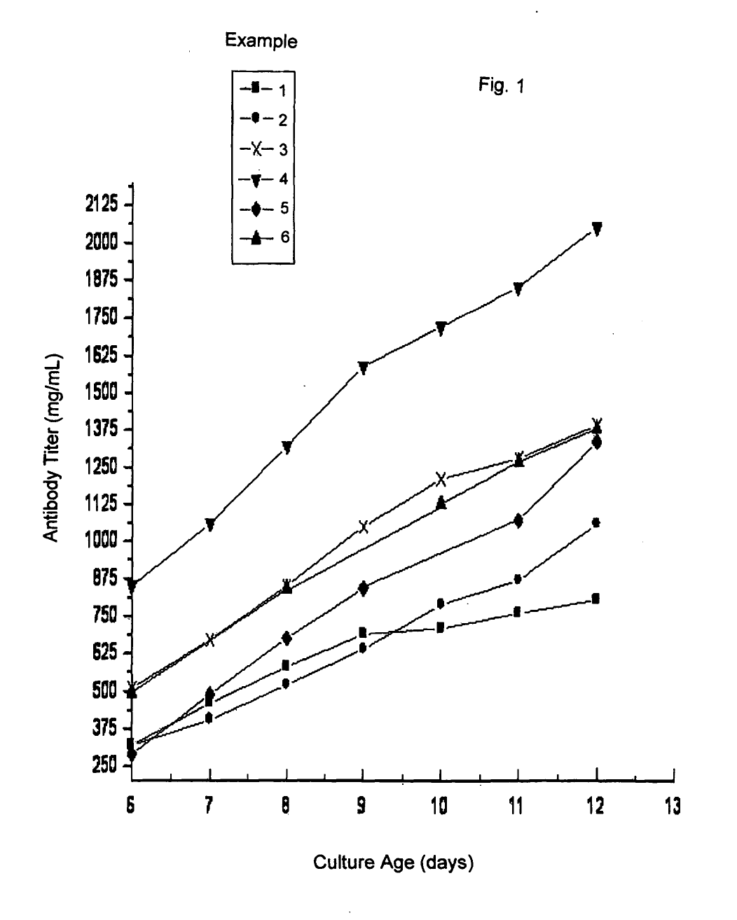

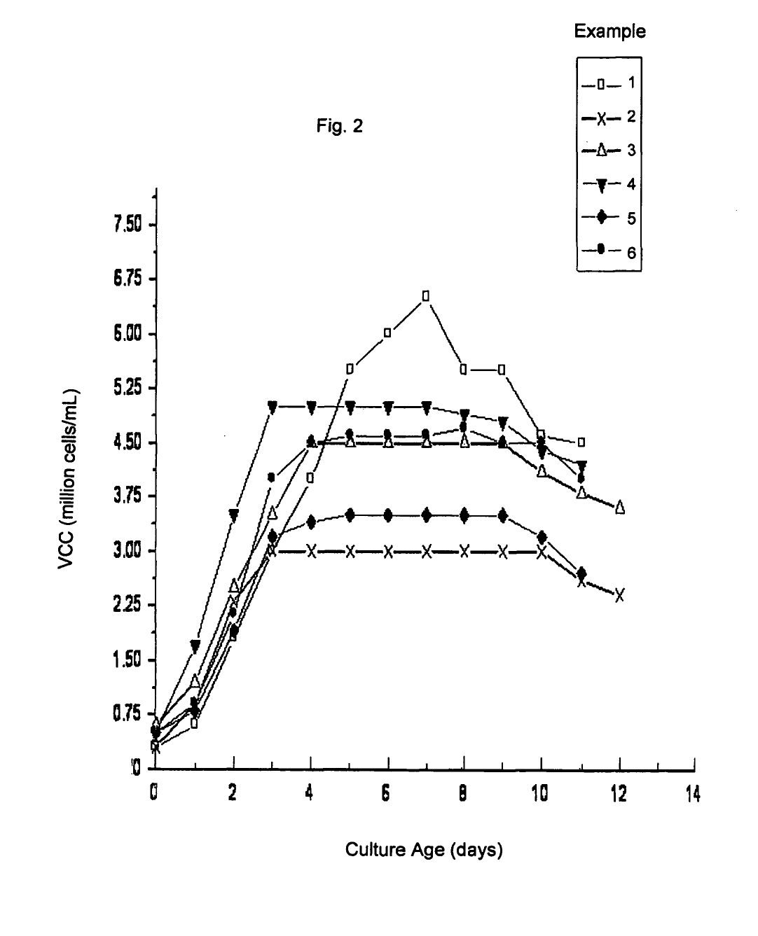

FIGS. 1 and 2 include illustrations of the “antibody titer” and viable cell count (VCC) profiles obtained by the procedure described in this example. The lines marked “1” represent the antibody titer and the VCC profile for this example.

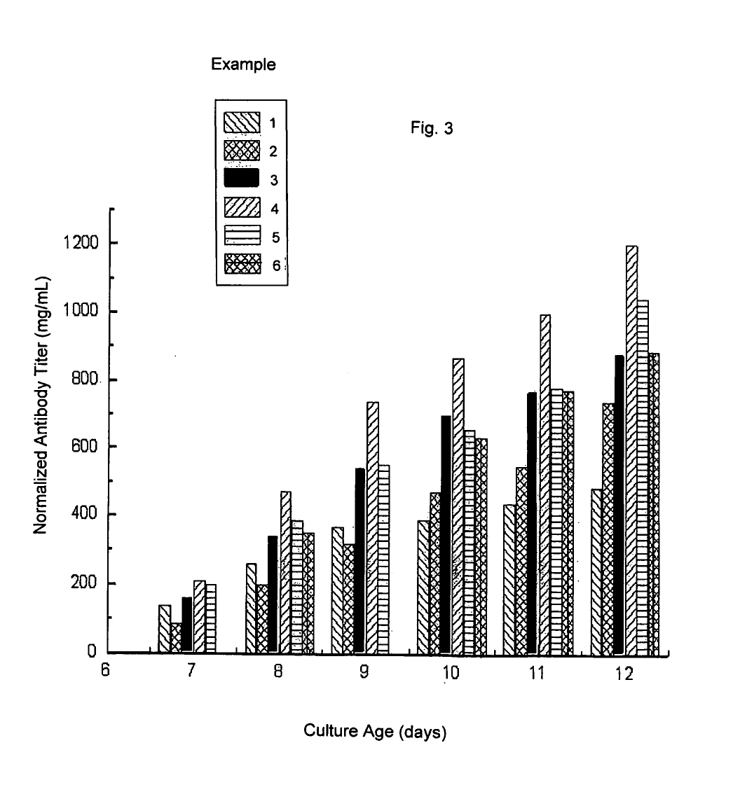

FIG. 3 is an illustration of the “normalized antibody titer,” being the difference between the antibody titer on the 6^{th} day and the following days. Bars show antibody titers for Examples 1-6, from day 7 to day 12.

### Example 2

An anti-VEGF antibody was cloned and expressed in a CHO cell line as described in U.S. Pat. No. 7,060,269. rCHO cells expressing antibody at a seeding density of 0.2-0.6 million cells/mL were grown in a PF CHO (HyClone®, Catalog no. SH30335 and SH30334) at 37° C. and an initial osmolality of 380-390 mOsm/kg. On attainment of optimum cell growth (Ivcc of 3 to 10 million cell-days/mL), the osmolality was increased to ≧450 mOsm/kg and ≦550 mOsm/kg and the temperature was lowered to 33° C. The culture was finally harvested after 288 hours or at 550% viablility. The resulting antibody yield was determined.

The antibody yield and cell viability are disclosed in Table 1.

FIGS. 1 and 2 include illustrations of the antibody titer and viable cell count profiles obtained by the procedure of this example. The lines marked “2” represent the antibody titer and the VCC profile obtained.

### Example 3

An anti-VEGF antibody was cloned and expressed in a CHO cell line as described in U.S. Pat. No. 7,060,269. rCHO cells expressing antibody at a seeding density of 0.2-0.6 million cells/mL were grown in a PF CHO (HyClone®, Catalog no. SH30335 and SH30334) at 37° C. and an initial osmolality of 320 mOsm/kg. During the growth phase, profile feeding of nutrients was done to increase the osmolality from 320 mOsm/kg to about 410 mOsm/kg (at 72 hours). On attainment of optimum cell growth (Ivcc of 3 to 10 million cell-days/mL), the osmolality was increased (by addition of nutrients and salts) to ≧450 mOsm/kg and ≦550 mOsm/kg and the temperature was lowered to 33° C. The culture was finally harvested after 288 hours or at greater than 50% viability and the resulting antibody yield determined.

The antibody yield and cell viability are disclosed in Table 1.

FIGS. 1 and 2 are illustrations of the antibody titer and viable cell count profiles obtained by the procedure of this example. The lines marked “3” represent the antibody titer and the VCC profile for this example.

### Example 4

An anti-VEGF antibody was cloned and expressed in a CHO cell line as described in U.S. Pat. No. 7,060,269. rCHO cells expressing antibody at a seeding density of 0.2-0.6 million cells/mL were grown in a PF CHO (HyClone®, Catalog no. SH30335 and SH30334) at 37° C. and an initial osmolality of 320 mOsm/kg. During the growth phase, profile feeding of nutrients was done to increase the osmolality from 320 mOsm/kg to 410 mOsm/kg (at 72 hours). On attainment of optimum cell growth (Ivcc of 3 to 10 million cell-days/mL), the osmolality was increased (by addition of nutrients and salts) to ≧450 mOsm/kg and ≦550 mOsm/kg and the temperature was lowered to 33° C. The culture was finally harvested after 288 hours or at greater than 50% viability and the resulting antibody yield determined.

The antibody yield and cell viability are disclosed in Table 1.

FIGS. 1 and 2 are illustrations of the antibody titer and viable cell count profiles obtained by the procedure of this example. The lines marked “4” represent the antibody titer and the VCC profile for this example.

### Example 5

An anti-VEGF antibody was cloned and expressed in a CHO cell line as described in U.S. Pat. No. 7,060,269. rCHO cells expressing antibody at a seeding density of 0.2-0.6 million cells/mL were grown in a PF CHO (HyClone®, Catalog no. SH30335 and SH30334) at 37° C. and an initial osmolality of 310-330 mOsm/kg. During the growth phase, profile feeding of nutrients was done to increase the osmolality to 390-410 mOsm/kg (at 72 hours). This was followed by further increase in osmolality to 460-480 mOsm/kg (by addition of nutrients and salts) and lowering of temperature to 33° C. On attainment of optimum cell density, the osmolality was further increased to ≧460 mOsm/kg and ≦550 mOsm/kg. The culture was finally harvested after 288 hours or at greater than 50% viability and the resulting antibody yield determined.

The antibody yield and cell viability are disclosed in Table 1.

FIGS. 1 and 2 are illustrations of the antibody titer and viable cell count profiles obtained by the procedure as described in this example. The lines marked “5” represent the antibody titer and the VCC profile for this example.

### Example 6

An anti-CD 20 antibody was cloned and expressed in a CHO cell line as described in U.S. Pat. No. 7,381,560. rCHO cells expressing antibody at a seeding density of 0.2-0.6 million cells/mL were grown in PowerCHO®-2 (Lonza, Catalog no. 12-771Q) at 37° C. and an initial osmolality of 310-330 mOsm/kg. During the growth phase, profile feeding of nutrients was done to increase the osmolality to 390-410 mOsm/kg (at 72 hours). This was followed by further increase in osmolality (by addition of nutrients and salts) to ≧450 mOsm/kg and ≦550 mOsm/kg and lowering of temperature to 33° C. The culture was finally harvested after 288 hours or at greater than 50% viability and the resulting antibody yield determined.

The antibody yield and cell viability are disclosed in Table 1.

FIGS. 1 and 2 are illustrations of the antibody titer and viable cell count profiles obtained by the procedure of this example. The lines marked “6” represent the antibody titer and the VCC profile for this example.

TABLE 1
Cell Viability and Antibody Concentration After 288 Hours.

| | Viability of Cells | | Final Concentration |
|---|---|---|---|
| Example | (%) | IVCC | (mg/L) |
| 1 | 30.58 | 69.6 | 805 |
| 2 | 88.9 | 31.7 | 1060 |
| 3 | 79.6 | 39.1 | 1390 |
| 4 | 86.8 | 42.5 | 2050 |
| 5 | 84.8 | 34.8 | 1335 |
| 6 | 71.2 | 38.0 | 1379 |

1. A cell culture process for protein expression in host cells comprising:
a) growing cells at one osmolar value and temperature;
b) subjecting cells to at least one higher osmolar value and temperature reduction; and
c) recovering the protein from the culture.

2. A process according to claim 1, wherein the cells are grown in step a) in an osmolality range from about 320-410 mOsm/kg.

3. A process according to claim 1, wherein cells in step b) are subjected to an osmolality range from about 380-450 m Osm/kg.

4. A process according to claim 1, wherein cells in step b) are subjected to an osmolality range greater than 450 mOsm/kg but less than 550 mOsm/kg.

5. A process according to claim 1, wherein cells in step b) are subjected to more than one higher osmolar value.

6. A cell culture process for protein expression in host cells comprising:
a) growing cells at an initial osmolar value and temperature;
b) culturing cells in at least one higher osmolar value;
c) subjecting cells to at least one increased osmolar value and reduction in temperature; and
d) recovering the protein from the culture.

7. A process according to claim 6, wherein cells are grown in step a) at an osmolality about 310-330 mOsm/kg.

8. A process according to claim 6, wherein the cells in step b) are cultured in an osmolality range from about 320-410 mOsm/kg.

9. A process according to claim 6, wherein the cells in step c) are subjected to an osmolality range greater than 450 mOsm/kg and less than 550 mOsm/kg.

10. A process according to claim 6, wherein at least one osmolality change is obtained by incremental addition of nutrients.

11. A cell culture process for expression of protein from host cells comprising:
a) growing cells at an initial osmolar value;
b) culturing cells in at least one higher osmolar value;
c) subjecting cells to at least one increased osmolar value and reduction in temperature;
d) subjecting cells to a further increased osmolar value; and
e) recovering the protein from the culture.

12. A process according to claim 11, wherein the cells are grown in step a) at an osmolality of about 310-330 mOsm/kg.

13. A process according to claim 11, wherein the cells are cultured in step b) in an osmolality range from 390-410 mOsm/kg.

14. A process according to claim 11, wherein the cells are cultured in step c) in an osmolality range from 460-480 mOsm/kg.

15. A process according to claim 11, wherein the cells are cultured in step d) in an osmolality range greater than 480 mOsm/kg and less than 550 mOsm/kg.

16. A process according to claim 11, wherein at least one osmolality change is brought about incremental addition of nutrients.

17. A process according to claims 1, 6 or 11, wherein a reduction of temperature is in the range of 2-7° C.

18. (canceled)

19. A process according to claim 1, 6, or 11, wherein cells are rCHO cell lines.

20.-21. (canceled)

22. A process according to claim 1, 6 or 11, wherein the protein is an antibody or an antigen binding fragment thereof or an Fc containing protein of mammalian origin.
